(19) United States
(12) Patent Application Publication
(10) Pub. No.: US 2026/0137284 A1
(43) Pub. Date: May 21, 2026

(54) SYSTEM AND METHOD FOR NON-INVASIVE MULTI-CHAMBER CARDIAC IDENTIFICATION

(71) Applicant: Aventusoft, LLC, Boca Raton, FL (US)

(72) Inventors: Brent E. Modzelewski, Delray Beach, FL (US); Claudia Iannini, Pembroke Pines, FL (US); Kaustubh Kale, Delray Beach, FL (US)

(73) Assignee: Aventusoft, LLC, Boca Raton, FL (US)

(21) Appl. No.: 19/445,986

(22) Filed: Jan. 12, 2026

Related U.S. Application Data
(63) Continuation-in-part of application No. 18/444,684, filed on Feb. 17, 2024.
Application No. 18/444,684 is a continuation-in-part of application No. 17/983,343, filed on Nov. 8, 2022.
Application No. 17/983,343 is a continuation-in-part of application No. 16/741,740, filed on Jan. 13, 2020, now Pat. No. 11,490,849.

(51) Int. Cl. A61B 5/0205; A61B 5/00; A61B 5/02; A61B 5/11; A61B 5/332; A61B 5/346; A61B 7/04

(52) U.S. Cl. CPC A61B 5/0205; A61B 5/02028; A61B 5/346; A61B 5/6833; A61B 5/1102; A61B 5/332; A61B 7/04

(57) ABSTRACT

A system and method for non-invasive identification of fiducials points of the events of the cardiac cycle, including but not limited to mitral valve closing (MC), aortic value opening (AO), aortic value closing (AC), mitral valve opening (MO), rapid systolic ejection (RE), rapid diastolic filling (RF), atrial systole (AS), isovolumic movement (IM), and isovolumic contraction (IC); additionally tricuspid valve closing (TC), pulmonic value opening (PO), tricuspid value closing (TC), pulmonic valve opening (MO). The system includes a device with multiple accelerometers, physiological electrical sensor, microcontroller for measuring and converting analog to digital signals and time deterministic synchronous sensor capture. The method includes seismocardiograph waveform fiducial detectors for peaks and troughs, phase detectors for mechanical and electrical signals, a heartbeat onset detector and one or more microprocessors to perform signal processing operations. Serving as a powerful, non-invasive system or method for quantifying myocardial performance.

### CROSS-REFERENCE TO RELATED APPLICATIONS

This application is a Continuation-in-part of U.S. patent application Ser. No. 18/444,684 filed on Feb. 17, 2024 entitled “System and method for non-invasive assessment of cardiovascular and pulmonary murmurs”, which is a Continuation-in-part of and claims priority to U.S. patent application Ser. No. 17/983,343 filed on Nov. 8, 2022 and a Continuation-in-part through U.S. patent application Ser. No. 16/741,740 filed on Jan. 13, 2020, which claims priority through U.S. patent application Ser. No. 15/397,138 filed on Jan. 3, 2017 which further claims the priority benefit of Provisional Application Nos. 62/274,766, 62/274,761, 62/274,763, 62/274,765, and 62/274,770 each of which were filed on Jan. 4, 2016 and each and all of the aforementioned applications claim priority thereto and incorporate such applications herein in their entireties.

### FIELD

This disclosure relates to the field of non-invasive physiological measurements and analysis, particularly for identifying multi-dimensional cardiac vibrational fiducials and evaluating events of the cardiac cycle as it relates to structural heart health, laying the foundational principles of cardiac time intervals.

### BACKGROUND

There are many methods used within the cardiac medical field to measure various aspects of the heart non-invasively. Electrocardiograms (ECG), phonocardiograms (PCG) and echocardiograms (ECHO) have historically been the standard of care. Emerging technologies, such as seismocardiograms (SCG) and gyrocardiograms (GCG), are beginning to gain attention due to similarities to phonocardiogram and to its simplicity of use in both medical facilities and home environments. Seismocardiograms have distinct advantages over phonocardiograms, having significantly increased measurement sensitivity when detecting valvular events. Typically, SCG measurements are captured using a single-axis, two-axis or three-axis accelerometer sensor. With the high degree of sensitivity also comes increased levels of noise not related to the valvular events of interest, resulting in drastic inaccuracies from patient-to-patient. These inaccuracies are compounded by variations in SGC signals caused by different disease states.

### SUMMARY

Healthy hearts as well as hearts with structural deficiencies can have varying morphologies compared to classic SCG signals, thereby making it extremely difficult, or even impossible, to properly identify the fiducials for these valvular events across the patient population using a fixed morphology model and a singular accelerometer sensor. Cardiac time intervals (CTIs), encompassing the various timing of the cardiac cycle, serve as a powerful, non-invasive means for quantifying myocardial performance. An evolving body of evidence demonstrates their significant value across a broad spectrum of cardiovascular diseases.

Foundational principles of cardiac time intervals: These begin by defining the cardiac cycle's temporal components, which are the precise sequential phases of the cardiac cycle that are foundational to understanding ventricular performance. The study of systolic time intervals (STI) provides a temporal description of this sequence, offering a measure of ventricular function that augments other metrics of myocardial performance. Due to their sensitivity and ease of measurement, STIs are particularly well-suited for assessing the heart's response to various physiological influences and pharmacological agents.

A detailed understanding of these intervals is critical for clinical application.

Mitral Valve Closure (MC), closure of mitral valve at end-diastole, provides physiological significance of Onset of ventricular systole; end of left ventricle (LV) filling. Abnormal delays or advance in closure time may indicate impaired diastolic function or elevated filling pressures.

Aortic Valve Opening (AO), opening of aortic valve when LV pressure>aortic pressure, provides physiological significance of the start of LV ejection, end of isovolumetric contraction. Delayed aortic opening can signal reduced contractility, correlates with afterload.

Aortic Valve Closure (AC), closure of aortic valve at end-systole (S2 sound), provides physiological significance of end of systole; onset of diastole. Delayed AC indicates high afterload, abnormal systolic unloading or altered aortic compliance.

Mitral Valve Opening (MO), opening of mitral valve when LV pressure falls below LA pressure, provides physiological significance of start of diastolic filling; end of isovolumetric relaxation. Delayed or shortened MO often suggests elevated left atrial pressure and reduced ventricular compliance.

Diastolic Filling Time (DFT), MO to next MC (diastole duration), provides physiological significance of available time for ventricular filling; can be shortened at high heart rates, reduced preload and elevated atrial pressure.

The Isovolumic Contraction Time (IVCT) is defined as the period of the cardiac cycle that begins with the closure of the mitral valve (MC) and concludes with the opening of the aortic valve (AO). During this phase, the ventricular muscle contracts, generating pressure within the chamber, but without any change in blood volume. This interval is a direct measure of the ventricle's ability to build force and overcome the pressure exerted by the aorta, known as afterload. A prolonged IVCT is a temporal marker of a struggling left ventricle that requires an extended period to generate sufficient pressure to open the aortic valve and initiate ejection, indicating increased afterload.

Following the ejection phase is the Isovolumic Relaxation Time (IVRT). This interval begins with the closure of the aortic valve and concludes with the opening of the mitral valve. During IVRT, the ventricle relaxes, and its pressure rapidly dissipates while its volume remains constant. As a critical marker of diastolic function, a prolonged IVRT indicates impaired myocardial relaxation, a sign of elevated filling pressures (i.e., congestion), high filling pressure.

Ejection Time (ET), which is the duration from the opening (AO) of the aortic valve to its closure (AC). This interval represents the period during which blood is ejected from the left ventricle into the aorta and serves as a key measure of systolic performance.

These individual metrics are physiologically significant, but their combination into a single index offers a more comprehensive assessment. The Myocardial Performance Index (MPI), also known as the Tei Index, is a composite measure defined as the sum of the isovolumic contraction and relaxation times, divided by the ejection time. This index provides a holistic view of global myocardial function by integrating both systolic and diastolic performance into a single, non-invasive metric. Its clinical value is rooted in its ability to detect cardiac dysfunction regardless of whether the primary impairment is in contraction or relaxation.

Cardiac time interval (CTI) Reference Ranges: Establishing Normative Data for Clinical Application.

For CTI measurements to be clinically useful, a robust set of reference ranges must be established to provide a baseline for interpretation. Normal reference ranges for CTIs have been published, but they are known to vary significantly by age and sex. For instance, studies have shown that IVRT and IVCT tend to increase with age in both sexes, while ET does not change significantly. A normal IVRT is typically cited at approximately 70 ms±12 ms, with values exceeding 110 ms indicating an abnormal myocardial relaxation and values below 60 ms suggesting a restrictive filling pattern. Establishing and using these demographic-specific reference ranges is crucial for accurate clinical assessment. FIG. 14 outlines the normal clinical range for key CTIs, such as, IVCT, ET, IVRT and MPI.

CTIs play a key role in disease surveillance.

### 1. Early Detection of Cardiac Dysfunction in Hypertensive and Ischemic Cardiovascular Disease Individuals

Hypertension is a prevalent condition that poses a significant risk for future ischemic cardiovascular diseases (ICVDs) such as stroke and ischemic heart disease. The progression from elevated blood pressure (BP) to a fulminant event is often an asymptomatic continuum, with organ damage serving as a critical intermediate stage. Conventional risk assessment tools and echocardiography frequently fail to detect these subtle, early changes in myocardial function.

CTIs provide a sensitive and quantifiable means to detect this asymptomatic damage. Research indicates that cardiac time intervals, particularly the IVRT/ET and MPI, are significantly impaired in hypertensive individuals when compared to non-hypertensive controls. This is notable because these cardiac impairments are often unrecognized by conventional echocardiography. The ability of CTIs to identify these “miniscule cardiac impairments” fundamentally changes the clinical paradigm. A patient with well-controlled BP but abnormal CTIs may still be at higher risk for future events, which could necessitate more aggressive pharmacological or lifestyle management. This effectively transforms CTI analysis into a tool for proactive, pre-emptive care rather than reactive management.

### 2. Prognosticating Future Ischemic Cardiovascular Events

Beyond early detection, CTIs possess powerful prognostic capabilities. Studies have demonstrated that the IVRT/ET and MPI are independent and potent predictors of future ICVDs, particularly in patients with known hypertension. These indices provide prognostic information that is incremental to conventional risk scores, such as the Framingham Risk Score and the European Society of Hypertension/European Society of Cardiology risk chart. A significant “dose-response relationship” has also been observed, wherein the severity of CTI impairment directly correlates with increasing blood pressure and left ventricular mass index (LVMI).

This capacity to quantify the specific relationship between BP severity, LV geometry (e.g., hypertrophy, remodeling), and CTI impairment allows for a more personalized approach to risk stratification. Rather than a generic risk score, a clinician can use CTI values to understand the specific physiological toll that hypertension is exerting on an individual's heart. For example, a patient with a high MPI and evidence of significant LV hypertrophy may require a different, more aggressive treatment strategy than a patient with a similar BP but a normal MPI. This moves clinical decision-making from broad, population-based guidelines to a more precise, data-driven, and patient-specific approach.

### 3. CTIs in Acute Coronary Syndrome (ACS) and Myocardial Infarction (MI)

The utility of CTIs extends to acute cardiac events. During an acute myocardial infarction (AMI), myocardial function deteriorates, and the CTIs change in predictable ways. The time required for the heart muscle to generate sufficient pressure to open the aortic valve (IVCT) and the time needed for the ventricle to relax (IVRT) both increase, while the time spent ejecting blood (ET) decreases. This combination of changes results in a significant increase in the Aortic valve opening.

The AO has been identified as a powerful, independent predictor of all-cause mortality in patients who have recently experienced an AMI. Conventional assessment of ventricular function after an MI traditionally focuses on Left Ventricular Ejection Fraction (LVEF). However, since an AMI affects both systolic and diastolic function, AO and also a composite index like the MPI provides a more comprehensive and superior prognostic tool. LVEF measures only systolic function, whereas the MPI, by combining IVCT and IVRT, captures the combined systolic and diastolic dysfunction that occurs during an infarction. This makes the MPI a more sensitive and predictive marker for adverse outcomes post-MI, guiding risk stratification and potentially informing more timely intervention in high-risk patients. The fact that the MPI can be reliably measured “irrespective of rhythm” is also a significant advantage in a clinical setting where arrhythmias such as atrial fibrillation are common. FIG. 15 outlines a comparative profile of disease states.

### 4. Clinical Applications Across the HF Spectrum

A notable strength of CTI monitoring is its applicability to both major HF phenotypes: heart failure with reduced ejection fraction (HFrEF) and heart failure with preserved ejection fraction (HFpEF). In HFrEF (reduced EF), with impairment of systolic function one expects prolonged IVCT and pre-ejection period (PEP), reduced LVET, and often the presence of a third heart sound or other acoustic markers of dilated ventricles. These changes can be tracked. For instance, as a patient's EF improves with therapy such as reverse remodeling with Guideline Directed Medical Therapy (GDMT), the Systolic Time Ratio should decrease toward normal, and IVCT may shorten. Conversely, an acute drop in EF would prolong these intervals. In HFpEF, EF is normal but filling is impaired and CTIs can reveal the diastolic etiology. A patient with HFpEF might have a relatively normal IVCT and ejection time, but an IVRT that is prolonged (relaxation abnormalities) and an S3 sound from abrupt deceleration of blood is present in early diastole. Mitral Valve Closure (MC) helps determine if filling phase is prematurely terminated, which may occur in stiff ventricles as seen in patients with HFpEF. Altered timing may suggest elevated LV end-diastolic pressure or impaired compliance. Delayed AO may indicate systolic dysfunction or increased afterload, which is common in HFrEF.

As HFpEF-related congestion worsens, IVRT may paradoxically shorten (when left atrial pressure rises to the degree in which the mitral valve opens sooner) thus trends in IVRT alongside heart sounds could signal transitions from pre-clinical stiffness to overt pulmonary edema, useful for monitoring diuretic response or evaluating diastolic dysfunction severity. Monitoring DFT allows optimization of heart rate, timing of diuretics, and beta-blockers. CTI monitoring could help assess chronotropic responses by examining how diastolic time shortens with heart rate. A recent study noted that exercise in HFpEF can drastically shorten diastolic filling time and raise filling pressures, explaining exercise intolerance.

Remote CTI monitoring can assist in therapy optimization. For example, when initiating a beta-blocker or titrating a vasodilator, one might expect improvements in CTIs (beta blockade might slightly prolong diastole but improve filling; vasodilators might shorten IVCT by reducing afterload). If CTIs worsen with prolongation of the IVCT, despite uptitration, it could indicate an inadequate response or need for alternative therapy. In device-managed patients, e.g. Cardiac Resynchronization Therapy (CRT) for Left Bundle Branch Block (LBBB), improvements in dyssynchrony would shorten IVCT and may even produce a crisper S1. If a persistently prolonged IVCT is present in a CRT recipient, it might prompt an echocardiographic reassessment of lead timing or represent an inadequacy of resynchronization therapy. Below is a comprehensive breakdown of CTIs for Chamber Performance, and how each plays a critical role in the management of heart failure, especially in distinguishing and tracking both HFpEF (preserved EF) and HFrEF (reduced EF). Key Systolic and Diastolic Events and Intervals in Cardiac Chamber Performance are shown in FIG. 16.

Beyond the acute setting, CTIs has shown promise in remote monitoring post-discharge and in chronic HF. Elevated CTIs can identify subclinical left ventricular dysfunction and congestion before patients become overtly symptomatic, enabling pre-emptive intervention. In a home monitoring context, patients perform daily or weekly measurements with HEMOTAG. Clinicians review CTI trends for warning signs of decompensation. A consistently rising IVCT or IVRT, or an increasing PEP/LVET ratio, might prompt an earlier up titration of diuretics or afterload reducers. CTIs can detect abnormalities days to weeks prior to symptom onset and help avert hospitalizations. The ability to involve patients in self-monitoring is an added benefit patients report feeling more engaged in their care when they can track their own “cardiac performance” metrics at home.

### Differentiating Cardiomyopathies with CTI Analysis

CTI analysis provides a unique window into the distinct pathophysiological mechanisms that underlie different forms of cardiomyopathy, enabling a more nuanced and accurate differential diagnosis.

### 1. Hypertrophic Cardiomyopathy (HCM)

Hypertrophic cardiomyopathy is a condition characterized by unexplained left ventricular hypertrophy in the absence of other causes like hypertension or valvular disease. A key pathology in HCM is diastolic dysfunction, which is defined by an impairment in the heart's ability to relax and fill properly. Research has consistently shown a significantly prolonged IVRT in patients with HCM when compared to control groups. This prolonged IVRT is a direct physiological consequence of the stiff, hypertrophied myocardium's inability to relax efficiently and is, therefore, a temporal marker for a fundamental biomechanical problem. The ability to quantify this relaxation abnormality via IVRT provides a non-invasive way to confirm a core pathological feature of the disease.

### 2. Restrictive Cardiomyopathy (RCM): the Paradox of a Shortened IVRT

In contrast to HCM, Restrictive Cardiomyopathy (RCM) is a rare condition characterized by increased myocardial stiffness that results in a rapid rise in ventricular pressure with only small increases in filling volume. Despite this stiff myocardium and its inherent diastolic impairment, the IVRT in RCM is often paradoxically shortened, typically measuring under 60 ms.

This paradoxical shortening of IVRT is a critical point for differential diagnosis and is a direct result of the unique pathophysiology of RCM. The stiff ventricular walls and elevated left atrial pressure cause the mitral valve to be forced open earlier than normal, thus truncating the isovolumic relaxation phase. Both HCM and RCM can present with signs of diastolic dysfunction and hypertrophy, making them difficult to distinguish. A physician relying solely on the presence of diastolic dysfunction might miss the subtle but critical difference. The contrasting IVRT findings prolonged in HCM and shortened in RCM provide a simple, non-invasive way to distinguish these two conditions. This underscores the power of CTI analysis to add nuance and precision to a challenging diagnostic problem.

### 3. Leveraging CTIS for Differential Diagnosis

The contrasting CTI patterns observed in various cardiomyopathies and other disease states illustrate their value as a differential diagnostic tool. For example, a prolonged IVCT points to increased afterload (as in aortic stenosis), whereas a prolonged IVRT suggests impaired myocardial relaxation (as in HCM) or increased filling pressure. The combined MPI, which is elevated in both conditions, would prompt a closer look at the individual time intervals to pinpoint the specific underlying pathology. This nuanced approach moves beyond a generic finding of “dysfunction” to a more specific identification of the biomechanical problem, guiding more targeted clinical management.

### Clinical Utility in Valvular Heart Disease and Arrhythmic Syndromes

### 1. Evaluating Ventricular Performance in Aortic Stenosis

Aortic stenosis (AS) is a valvular disease that imposes a significant pressure overload, or afterload, on the left ventricle. This mechanical burden directly affects the cardiac time intervals. A prolonged IVCT is a direct physiological consequence, as the ventricle needs more time to generate the high pressure required to open the narrowed aortic valve and eject blood.

While the severity of AS is conventionally graded using metrics like peak velocity, mean pressure gradient (MPG), and aortic valve area (AVA), these can be inconsistent, particularly in patients with low stroke volume. In such cases, the area under the curve (AUC) of isovolumetric contraction, a measure of the mechanical work performed during this phase, offers a valuable alternative. The AUC of isovolumic contraction has been shown to correlate with ejection fraction and total ventricular work, providing a measure of the afterload mismatch that is independent of conventional flow-based metrics. This moves the diagnostic focus from the valve itself to the ventricle's physiological response, which is a more accurate reflection of the patient's overall health and prognosis.

### 2. The Impact of CTIs on the Management of Mitral Regurgitation

Cardiac time intervals are not only valuable for diagnosis but can also be leveraged for therapeutic purposes. Mitral regurgitation (MR), a condition where the mitral valve leaks, is a common complication of heart failure. The timing of MR is closely related to the pressure differential between the left ventricle and the left atrium. Research has demonstrated a direct relationship between the programming of the atrioventricular (AV) interval in patients with cardiac resynchronization therapy (CRT) and the onset of MR.

This finding is a direct example of how understanding CTI physiology can be used therapeutically. By adjusting the AV pacing interval to optimize the timing of left atrial contraction and subsequent left ventricular pressure buildup, clinicians can modulate the onset of MR. CTI analysis provides the real-time feedback necessary to fine-tune these complex device settings, which can lead to a reduction in MR and an improvement in cardiac efficiency, thereby alleviating a common and morbid complication of advanced heart disease.

### 3. The Long Qt Syndrome (LQTS): a Case Study in Electrical-Mechanical Coupling

The Long QT Syndrome (LQTS) is a congenital or acquired disorder that causes a prolongation of the QT interval on the ECG. This abnormal electrical timing reflects a delay in ventricular repolarization, often due to ion channel dysfunction, and puts patients at risk for fatal arrhythmias such as Torsades de Pointes.

While the QT interval is an electrical CTI, its abnormal prolongation creates a temporal vulnerability window during which the heart is susceptible to life-threatening arrhythmias. This highlights the fundamental link between the heart's electrical and mechanical systems. An electrical instability, if uncorrected, can lead to a chaotic mechanical cascade culminating in ventricular fibrillation and sudden cardiac death. CTI analysis, in this context, serves not only to identify the electrical anomaly via ECG but also to inform the clinical response to prevent the subsequent catastrophic mechanical failure.

### 4. A Novel Application: CTIS in Arrhythmic Syndromes

Arrhythmias, such as atrial fibrillation (AFib) and atrial flutter, are characterized by abnormal electrical signals that cause the heart to beat too fast, too slow, or erratically. In atrial fibrillation, chaotic electrical signals cause the atria to quiver, leading to an “irregularly irregular” rhythm and inefficient contraction. Similarly, atrial flutter is caused by a rapid electrical circuit in the right atrium, leading to atrial contractions of 240-340 beats per minute. These conditions can cause blood to pool in the atria, increasing the risk of stroke and potentially leading to heart failure.

Assessing heart function in patients with irregular rhythms presents a significant challenge for conventional metrics that rely on consistent cardiac cycles. The Myocardial Performance Index (MPI), however, offers a powerful solution as it provides independent prognostic information regardless of the patient's rhythm. A study on patients with acute myocardial infarction demonstrated that the MPI was a simple, reproducible measure that provided independent prognostic information even in patients with atrial fibrillation. This is a crucial finding, as the MPI's ability to integrate both systolic and diastolic performance makes it a more reliable predictor of adverse outcomes than separate measures in these complex cases.

Another time-based metric, the Visually Assessed Time Difference between Mitral Valve and Tricuspid Valve Opening (VMT) score, has emerged as a valuable tool for patients with atrial fibrillation. This score visually assesses the temporal relationship between the opening of the mitral valve and the tricuspid valve, with a score of VMT≥2 indicating that the mitral valve opens first. This specific finding is strongly correlated with elevated left ventricular filling pressure and more severe heart failure symptoms. The VMT score's clinical value is particularly high in AFib patients because their monophasic left ventricular inflow pattern can make it difficult to use conventional Doppler parameters for assessing filling pressure.

The utility of CTI analysis also extends to patients with Left Bundle Branch Block (LBBB), a cardiac conduction disorder where the electrical impulse to the left ventricle is delayed or blocked. This condition causes the right ventricle to contract before the left, leading to mechanical dyssynchrony. This abnormal activation sequence prolongs the total isovolumic time (IVT), which can be measured as an indicator of myocardial health. Since LBBB is often associated with underlying heart conditions and a worse prognosis, the ability to measure this conduction abnormality and its hemodynamic consequences non-invasively provides valuable clinical insight.

### 5. Benefits of Remote CTI Monitoring

Remote monitoring of CTIs offers several potential benefits

Early Warning of Decompensation: By tracking subtle changes in cardiac function (before overt weight gain or symptoms), CTI monitoring can alert clinicians to impending decompensation. As noted, prolonged CTIs correlated strongly with high BNP in acute HF, and anecdotal use showed detection of abnormalities before symptom onset. This could translate to timely intervention (e.g. adjust diuretics) and prevention of hospitalizations.

Objective Therapy Guidance: CTIs provide quantitative, beat-to-beat information on how the heart is responding. This could guide titration of medications. For instance, an ACE inhibitor might be increased until no further improvement in PEP or IVRT is observed, indicating an optimal hemodynamic effect. In contrast, patient-reported symptoms or weights can be quite variable and subjective.

Applicability to Diverse HF Populations: CTI monitoring is useful in HFrEF, HFpEF, and even other cardiac conditions (valve disease, post-myocardial infarction dysfunction, etc.) because it measures fundamental aspects of cardiac cycle timing. This is unlike some telemonitors that focus solely on fluid (more relevant to congestion) or arrhythmia (relevant to certain subsets).

Non-Invasive and Patient-Centric: Patients can perform daily 30-second readings themselves, at home, without blood draws or imaging. Empowering patients to be active participants in their care. It also reduces the need for frequent clinic visits just for surveillance, which is important given the burden of HF clinic follow-ups.

CTI reference ranges by age and sex are illustrated in FIG. 17.

Cardiac time interval-based surveillance represents a paradigm shift in cardiology, moving beyond traditional metrics to provide a more sensitive and holistic view of myocardial health. This innovation demonstrates its utility in the early detection of sub-clinical dysfunction in hypertension, the nuanced differential diagnosis of cardiomyopathies, and the comprehensive assessment of ventricular loading in valvular disease. The ability to this innovation to measure these intervals with high precision addresses previous limitations related to reproducibility and operator skill. The integration of CTI analysis into routine clinical practice holds the promise of a more proactive and personalized approach to the management of cardiovascular disease, ultimately improving patient outcomes.

Various heart movements and valve events, including valve openings and closings, result in a set of interfering mechanical vibrations, which have proven to be extremely difficult to deconvolve into specific events of the cardiac cycle. Multiple convoluted vibrations from heart movements are the key reasons why technologies like SCG, PCG and GCG are rarely considered for high accuracy medical diagnosis of structural heart conditions. Transthoracic vibrations measured from a single point on the chest will experience vibrational signals that are a composite of multiple physical events occurring both inside and outside the chest cavity, proving impossible to separate these different simultaneous or near-simultaneous events into accurate events related to the cardiac cycle.

The embodiments herein solve the problem of identifying SCG signal signatures related to events of the cardiac cycle despite the presence of extraneous vibrational data whether these extraneous vibrations are from systematic physiological occurrences, externally generated sounds or non-periodic bodily movements. The embodiments entail placement of two or more vibrational or accelerometer sensors of a fixed known distance from each other, affixed externally, with electrodes or adhesive pads, to a patient's chest. Leveraging physiological signal phase differences using N number of time-synchronized seismocardiograms from different but distinct physical locations, this method can identify events such as the heart valve open and closing times, while effectively ignoring other unrelated sounds.

Increasing N sensors, linearly increases the number of locational events that can be accurately measured where N-1 represents the number of locations that can be measured. For example, N=3 can measure two (2) simultaneous events discerning their respective vibrational origins.

Although multi-axis accelerometers may be used to increase sensitivity, the embodiments herein are robust enough to only require a single-axis of measurement placed accordingly.

This innovation allows for a paradigm shift toward the integration of cardiac time interval-based surveillance as a standard of care, advocating for a more nuanced, proactive, and individualized approach to the management of cardiac, metabolic and pulmonary diseases.

### BRIEF DESCRIPTION OF THE DRAWINGS

The accompanying drawings, which are incorporated herein and form part of the present disclosure, illustrate exemplary embodiments of the invention and, together with the detailed description, serve to explain the principles, structure, and operation of the disclosed systems and methods. The drawings are provided for illustrative purposes only and do not limit the scope of the embodiments.

FIG. 1 illustrates standard auscultation points locations relative to front view of the ribcage;

FIG. 2 illustrates internal heart valve locations relative to front view of the ribcage;

FIG. 3A illustrates example device shape and relative dimensions of two sensors in accordance with the embodiments;

FIG. 3B illustrates example device shape and relative dimensions of three sensors in accordance with the embodiments;

FIG. 3C illustrates example device shape and relative dimensions of four sensors in accordance with the embodiments;

FIG. 3D illustrates example device shape and relative dimensions of five sensors in accordance with the embodiments;

FIG. 4A illustrates device with two sensor locations applied to chest cavity with relation to heart valve locations in accordance with the embodiments;

FIG. 4B illustrates device with two sensor locations applied to chest cavity with relation to heart valve locations in accordance with the embodiments;

FIG. 4C illustrates device with two sensor locations applied to chest cavity with relation to heart valve locations in accordance with the embodiments;

FIG. 4D illustrates device with two sensor locations applied to chest cavity with relation to heart valve locations in accordance with the embodiments;

FIG. 5 captured data of classic three channel SCG signal with illustration of an ECG signal only indicating onset of heartbeat in accordance with the embodiments;

FIG. 6 captured data of a normal sternum channel SCG signal with confounded SCG signals on aortic and pulmonic with illustration of an ECG signal only indicating onset of heartbeat in accordance with the embodiments;

FIG. 7 captured data of normal sternum and aortic channel SCG signals with confounded SCG signal on pulmonic with illustration of an ECG signal only indicating onset of heartbeat in accordance with the embodiments;

FIG. 8 captured data of three channels SCG signals with confounded SCG peaks with illustration of an ECG signal only indicating onset of heartbeat in accordance with the embodiments;

FIG. 9 captured data of SCG exhibits low amplitude SCG due to multiple heart conditions for patient with illustration of an ECG signal only indicating onset of heartbeat in accordance with the embodiments;

FIG. 10 illustrates heart anatomy and valves;

FIG. 11 illustrations generic time synchronized SCG, PPG and GCG signal comparison with marked fiducials points with illustration of an ECG signal only indicating onset of heartbeat in accordance with the embodiments;

FIG. 12 is a flowchart of actionable CTI based indication for guided treatment for heart failure management in accordance with the embodiments;

FIG. 13 is a graph of precise intracardiac hemodynamics with CTIs: Preload, afterload, and contractility for earlier intervention in accordance with the embodiments.

FIG. 14 a table of key CTI definitions and metrics.

FIG. 15 is a table of comparative CTI profiles in disease states.

FIG. 16 is a table of clinical relevance of CTIs in patients with HFrEF and HFpEF.

FIG. 17 CTI reference ranges by age and sex.

### DETAILED DESCRIPTION

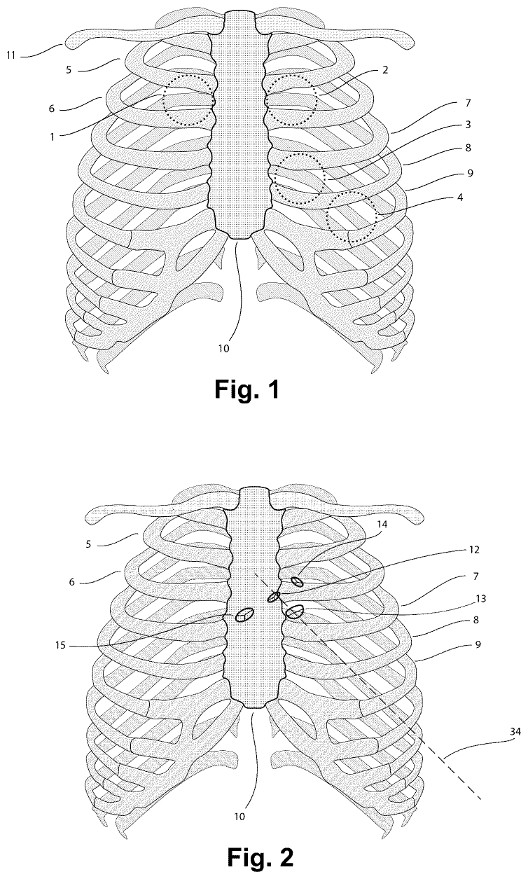

The rib cage is depicted as if you were facing the patient, with standard auscultation points are on FIG. 1, with the Aortic Auscultation point 1, Pulmonic Auscultation point 2, Tricuspid Auscultation point 3 and Mitral Auscultation point 4. Aortic Auscultation point 1 is located on the right side of the rib cage between the second rib 5 and the third rib 6. Pulmonic Auscultation point 2 is located on the left side of the rib cage between the second rib 5 and the third rib 6. Tricuspid Auscultation point 3 is located on the left side of the rib cage between the fourth rib 7 and the fifth rib 8. Mitral Auscultation point 4 is located on the left side of the rib cage between the left rib 8 and the sixth rib 9. The Aortic Auscultation point 1 and the Pulmonic Auscultation point 2 are evenly placed to either side of the sternum 10, which is the centerline of the chest.

The rib cage is again depicted on FIG. 2, showing the physical position of the heart values, Aortic Valve 12, Mitral Valve 13, Pulmonary Valve 14 and Tricuspid Valve 15. It becomes evident that the auscultation points on FIG. 1 that are used when listening to heart with a traditional stethoscope do not align with heart valve locations of FIG. 2. In the embodiments, the valve locations 12 13 14 15 of FIG. 2 are important for accurately identifying events of the cardiac cycle.

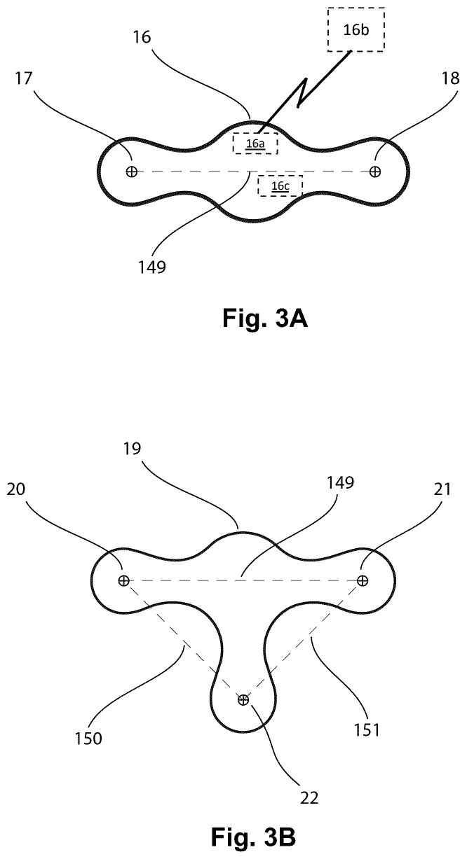

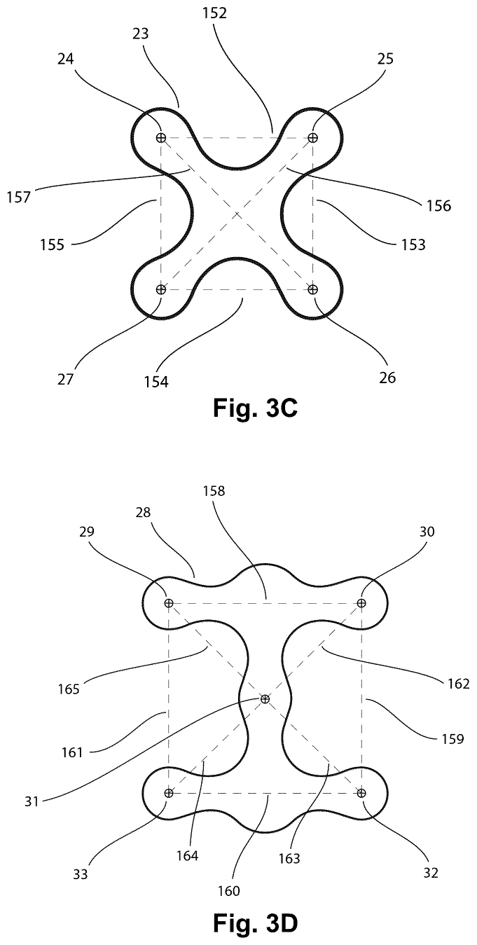

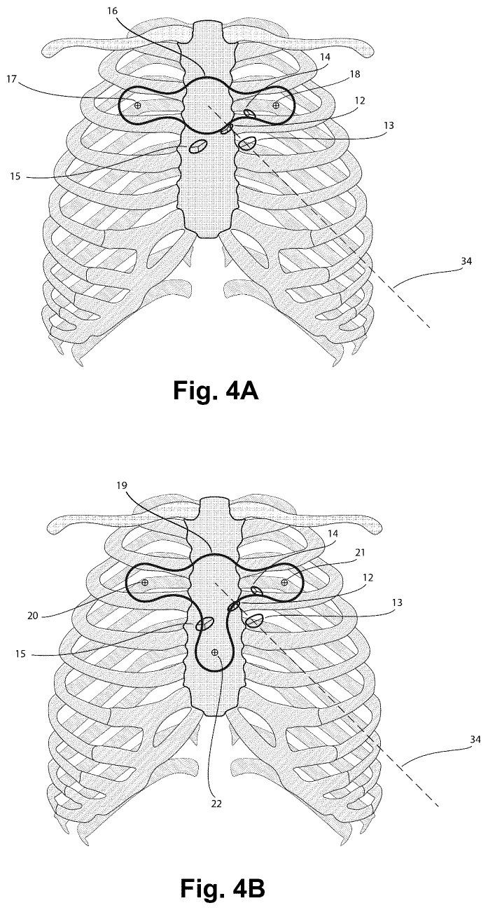

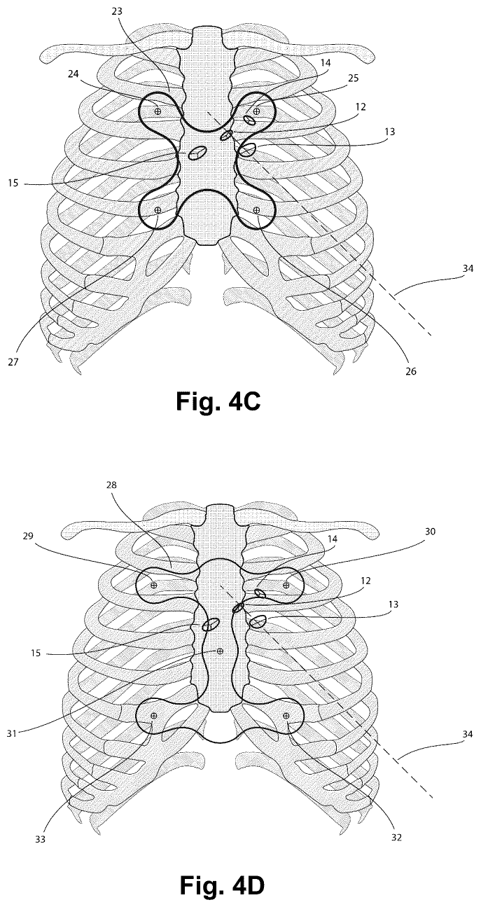

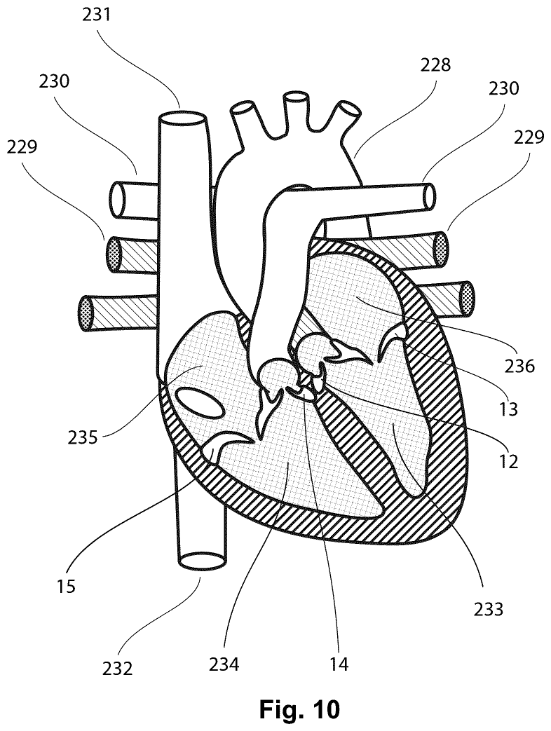

A device 16 with two or more vibrational sensors (17, 18), such as accelerometers, of a fixed and known distance 149 from each other on FIG. 3A configured to measure time-synchronized vibrations coming through the thoracic cavity, is used to identify and isolate multi-chamber cardiac events using vibrational frequency, multi-dimensional wave amplitude (peaks/troughs), and relative vibrational phase differences between sensors. The accelerometers are time-synchronized with an electrical sensor 16c for detecting the onset of the heartbeat, which coincides with the Q fiducial point of an ECG. A device 16 with two accelerometers on FIG. 3A placed horizontally on the chest across the vertical centerline of the sternum on FIG. 4A can be used to identify events that occur on each side of the heart. Note that the electrical sensor 16c should be connected to at least two points, but in some embodiments the electrical sensor 16c can be a “single-lead” ECG, but still having 2 electrical points to close the circuit. A device 19 with three accelerometers on FIG. 3B placed in a triangular-shape where two time-synchronized vibration detection channels or sensors 20 21 are placed horizontally at the top of the heart and across the centerline of the chest (across the heart), while the third vibrational sensor 22 is placed on the center line toward bottom of heart on the sternum 10 as shown on FIG. 4B, can be used to identify events on each side of the heart with more robust identification that can occur in as few as a single heart beat by analyzing combination of sensors in pairs. A device 23 using four accelerometers on FIG. 3C, where the sensors can be placed such that each individual sensor 24 25 26 27 as shown on FIG. 4C can read vibrations from each of the four chambers on FIG. 10, and combinations of sensors can be combined to isolate more cardiac events. With five accelerometers on FIG. 3D, the sensors can detect events from all four chambers as shown in FIG. 4D, while rejecting additional extraneous unrelated vibrations. Each of the embodiments can utilize one on more processors for signal processing and other processing of vibrational and/or electrical signals either on-board on the respective devices (16, 19, 23 or 28) or in combination with a remote device. For example, for illustration purposes, an on-board or local processor 16a can process signals from sensors 17, 18 and/or 16c or a combination of processors including the processor 16a and a remote processor 16b can process the signals from the sensors 17, 18, and/or 16c. The other embodiments can also include processors, but are not shown to simplify the drawings. Further note that the electrical sensor 16c does not necessarily need to be in the same pre-determined position relative to the vibration sensors. There is some flexibility on such placement. However, the vibrational sensors need to be time synchronized so the electrical sensor is on the same timing as the vibrational sensors. The vibration sensors are pre-arranged in space or distance so the system can see the difference in sound travel in intensity and time based on the location. The electrical signal travels fast enough that its location is not important or as important.

In one example, two sensors 17 18 on FIG. 3A can detect Mitral Valve 13 and Aortic Valve 12 on FIG. 4A for openings and closings. The timing of Tricuspid Valve 15 on FIG. 2 events nearly coincide with the Mitral Valve 13 events. Whereas the time of the Pulmonic Valve 14 on FIG. 4A and Aortic Valve 12 events nearly coincide with each other. Two sensors can help separate the small timing differences in Mitral Valve 13 and Tricuspid Valve 15 events, and the Aortic Valve 12 and Pulmonic Valve 14 events. Two sensors can detect the origination of the sound side to side in the body.

Three sensors 20 21 22 on FIG. 3B can detect Mitral Valve 13 and Aortic Valve 12 on FIG. 4A during openings and closings with a single heartbeat, where two sensors would need more heartbeats to ascertain a peak/trough identification by separating non-periodic extraneous vibrations. Three sensors can reject both periodic and non-periodic extraneous vibrations due to the extra channel of measurement, as well as identify signals that originate in a vertical direction.

Four sensors 24 25 26 27 as shown on FIG. 3C can begin to separate additional simultaneous events from the mechanical cycle, coronary artery sounds, or simultaneous pulmonary sounds.

With sensors placed near the aorta 228 as shown on FIG. 10 or the pulmonary veins 229 as shown on FIG. 10, detection of heart conditions such as aortic stenosis and mitral regurgitation can be adeptly identified through evaluation of resulting specific vibrations due to blood flow turbulence, separating those specific vibrations from heart valve vibrations. With additional sensors placed near the two branches of the Pulmonary Artery 230 on FIG. 10 or the superior vena cava 231 as shown on FIG. 10 or inferior vena cava 232 as shown on FIG. 10, such a system can perform a detection of heart conditions such as Arrhythmogenic Right Ventricular Cardiomyopathy (ARVC) and Cor Pulmonale.

Increasing the number of sensors on the device improves the directional ability to pinpoint the origin of a specific mechanical vibration and isolate it from other simultaneous heart vibrations. As N number of sensors, with fixed known placement on the device increases and approaches infinity, a time-lapse animation can be constructed showing the entire movement of the heart non-invasively.

A frequency filter, such as low pass, band pass or high pass, is used to isolate valvular events based on specific valve and its known frequency response for the valve physical size and direction of which heart chamber it is opening into.

Traditional signal processing, artificial intelligence or machine learning algorithms can be used as peak and trough detectors to isolate valvular events based on the sensor location and its relative direction of the valve opening and closing. The peak and trough detectors must be used in combination with a phase detector to isolate the location of where the event took place.

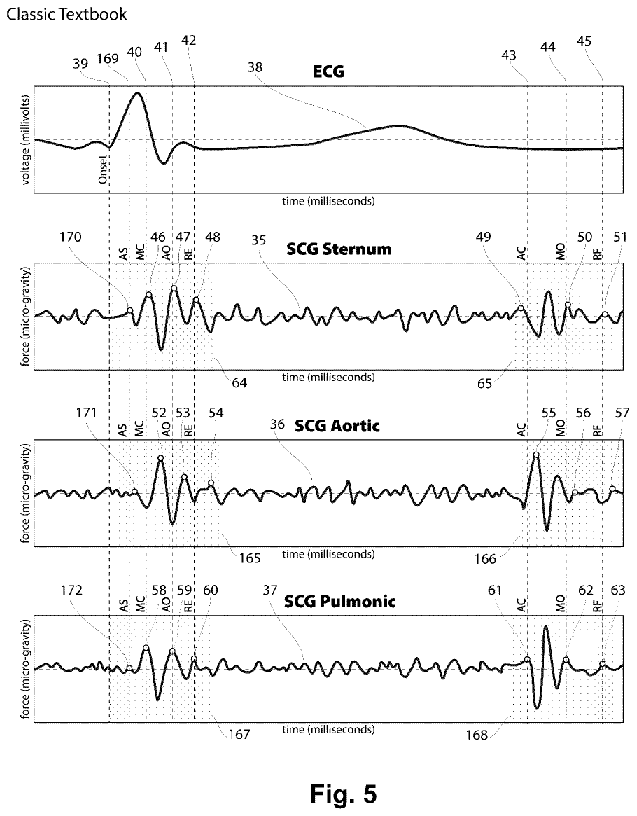

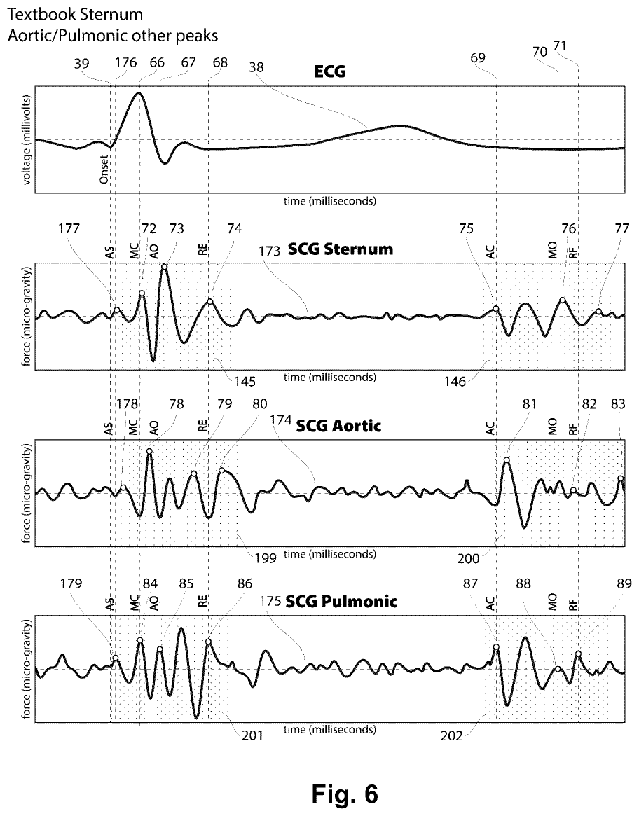

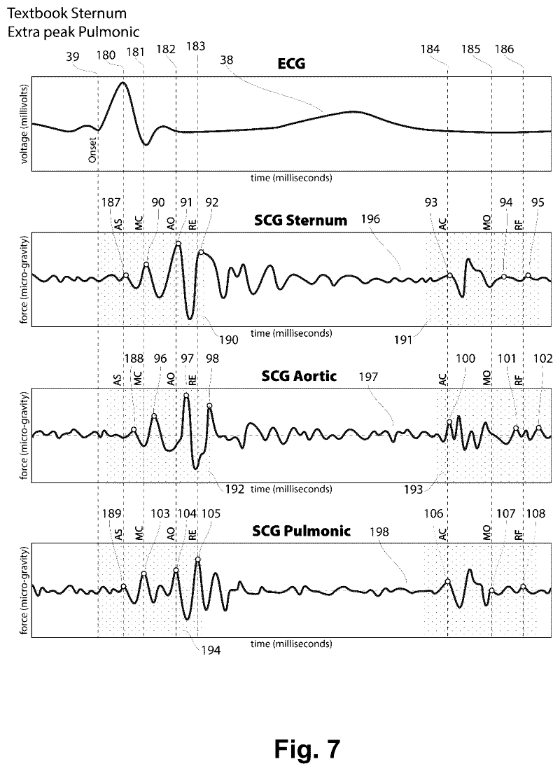

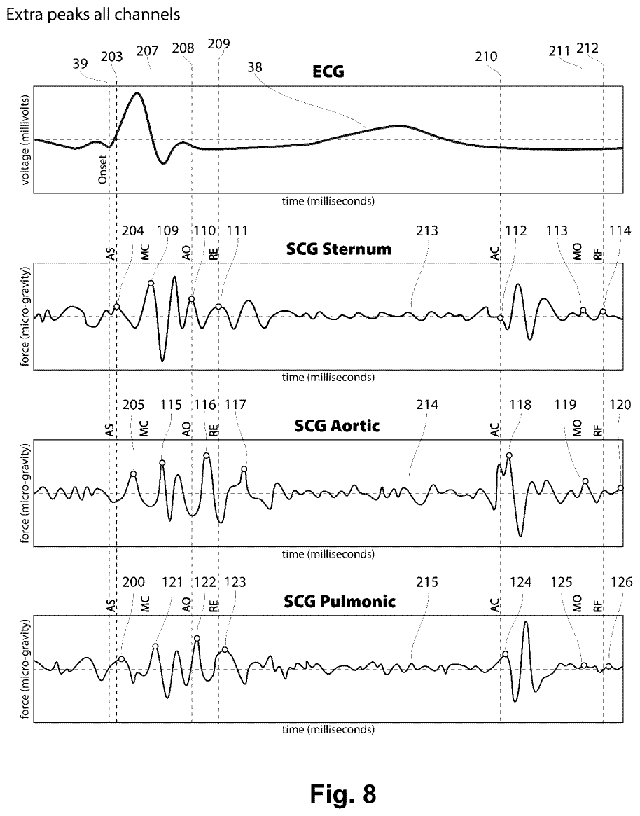

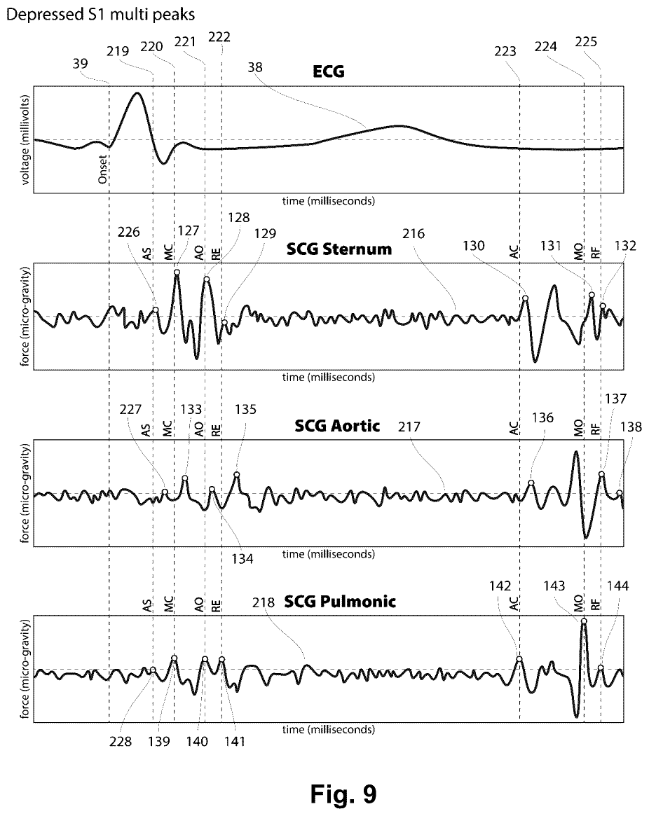

The three sensor locations 20 21 22 on FIG. 4B are located to target the Aortic Auscultation point 1, Pulmonic Auscultation point 2, and the Sternum 10 (see FIG. 1), respectively. For notation purposes, the different sensor channels are labeled SCG Sternum, SCG Aortic and SCG Pulmonic on FIG. 5, FIG. 6, FIG. 7, FIG. 8, FIG. 9, along with an electrical sensor channel labeled ECG to depict the onset of the heartbeat. The ECG signal 38 on FIG. 5, FIG. 6, FIG. 7, FIG. 8, FIG. 9 is only used to illustrate the onset 39 of the heartbeat. For graph-to-graph comparison purposes, the same ECG signal is depicted on FIG. 5, FIG. 6, FIG. 7, FIG. 8, FIG. 9 and is not suggestive that all patients have the same ECG, in fact, patients have different ECG signals and can vary significantly with heart conditions.

A classic textbook SCG signal 35 on FIG. 5 focusing on the Sternum SCG 35 only, is shown with Mitral Close 40 sternum fiducial 46 occurring as first peak after ECG signal 38 onset 39, Aortic Open 41 sternum fiducial 47 as largest peak in the S1 sound 64, followed by the Rapid Ejection 42 identified by the next sternum peak 48. The classic textbook SCG signal continues with S2 65 with clearly identified Aortic Close 43 at sternum fiducial 49, Mitral Open 44 at sternum fiducial 50 and Rapid Filling 45 at sternum fiducial 51. This classic textbook SCG signal for healthy hearts recorded from the sternum position is the basis of prior art's detection algorithms tuned to this fixed morphology. The classic textbook signals morphology can be seen at other locations, such as Aortic signal 36 and Pulmonic signal 37, where the Mitral Close 40 fiducials 52 58, Aortic Open 41 fiducials 53 59, Rapid Ejection 42 fiducials 54 60, Aortic Open 43 fiducials 55 61, Mitral Open 44 fiducials 56 62 and Rapid Filling 45 fiducials 57 63 respectively. Looking at Mitral Close 40 for all these time-synchronized channels 35 36 37, as an example, demonstrates the vibration of the Mitral Close 44 event reaching the SCG Pulmonic first at 58, followed by SCG Sternum 46 and lastly SCG aortic 52.

For all the events of the cardiac cycle, it is clearly shown in FIG. 5 that a time-phase shift exists between different channels based on a known fixed distance between channel sensors. With multiple channels at different locations, the embodiments herein has improved accuracy by selecting the earliest signal measured for the cardiac event, whereas the prior art has a built error that is variable between patients and device placement. The physiological mechanism for the phase-shift is shown in FIG. 4B for a triple-sensor device 19 where first sensor 21 and second sensor 22 are approximately equidistant from the Mitral Valve 13 as seen by the bisecting angle 34, and the third sensor 20 is further away. Although the example shown in FIG. 5 indicates first detection on the SCG Pulmonic 37, this can change to first detection on the SCG Sternum 35 based on patient device placement and individual heart anatomy. However, this embodiment is resilient to that external variation by selecting the first signal identifying the event.

A textbook sternum (only) SCG signal 173 in FIG. 6 is shown with Mitral Close 66 sternum fiducial 72 occurring as first peak after ECG signal 38 onset 39, Aortic Open 67 sternum fiducial 73 as largest peak in the S1 sound 145, followed by the Rapid Ejection 68 identified by the next sternum peak 74. The textbook sternum (only) SCG signal continues with S2 146 with clearly identified Aortic Close 69 at fiducial 75, Mitral Open 70 at fiducial 76 and Rapid Filling 71 at fiducial 77.

Just like the classic textbook SCG signal in FIG. 5, the textbook sternum SCG signal in FIG. 6 is for healthy hearts recorded from the sternum position with existing technology tuned to this fixed morphology. However, unlike the classic textbook signals on FIG. 5, the textbook sternum on FIG. 6 exhibits different morphologies at other locations, such as Aortic signal 174 and Pulmonic signal 175, where on FIG. 7 there are a greater number of S1 peaks/throughs 194 compared to classic textbook SCG. Looking at Mitral Close 66, as an example, demonstrates the vibration of the event reaching the SCG Pulmonic first at 84, followed by SCG Sternum 72 and lastly SCG Aortic 78. Although the example shown on FIG. 6 indicates first detection on the SCG Pulmonic 175, this can change to first detection on the SCG Sternum 173 based on patient device placement or heart anatomy. A single sensor would be unable to recognize different morphologies at multiple locations, thereby increasing the error in identifying the proper fiducial with presence of these extra vibrations.

Extra peaks/throughs can exist on all channel data as seen on FIG. 8 between 109 and 110 on SCG Sternum signal 213, between 155 and 116 on SCG Aortic signal 214, between 200 and 121 on SCG Pulmonic Signal 215, and between 121 and 122 on SCG Pulmonic Signal 215 during the S1 heart sound. Through analysis of the phase-shift between the channel sensors based on their relative position, it becomes clear peaks 109 121 115 should be selected as Mitral Close event 207 with the event occurring on SCG Sternum signal 213 first, and peaks 110 122 116 should be selected as Aortic Opening event 208. This pattern continues for the remaining peaks 112 124 118 for Aortic Closing event 210 and peaks 113 125 119 for Mitral Opening event 211. In this example, without accounting for the fixed relative positioning 149 150 151 of the three-sensor device 19 on FIG. 3B and the sensor relation to the heart values 12 13 on FIG. 4B the phase-difference would not be possible to be used to select the correct fiducial points.

For certain heart conditions, S1 sounds can become damped as seen on FIG. 9 for both the SCG Aortic 217 and SCG Sternum signals 218. The depressed signals still hold vital information for detecting cardiac events. The fiducial points become clear when evaluating the phase difference in the signals, and again in this example the SCG Pulmonic signal 218 occurs first producing fiducial points 139 140 142 143 equating to the main valvular events Mitral Closing 220, Aortic Opening 221, Aortic Closing 224 and Mitral Opening 224 respectively. The SCG Sternum signal 216 follows with corresponding fiducials shifted from the SCG Pulmonic signal 218. The SCG Aortic signal 217 follows suit, with all three channels in agreement for proper cardiac events.

Once fiducial points of the events of the cardiac cycle, such as Mitral Valve Closing (MC), Aortic Valve Opening (AO), Aortic Valve Closing (AC) and Mitral Valve Opening (MO) are identified, time intervals known as Cardiac Time Intervals (CTI) can be calculated using simple mathematical operations. The fiducials MC, AO, AC and MO are represented in the time from the electrical onset (Q point on the ECG) to the fiducial itself. A well-known CTI is the Isovolumic Contraction Time (IVCT) is calculated as IVCT=AO−MC. Other well-known CTI's are Isovolumic Relaxation Time (IVRT) calculated as IVRT=MO−AC and Left Ventricular Ejection Time (LVET) calculated as LVET=AC−AO. Fiducial points such as Atrial Systole (AS), Isotonic Contraction (IC), Isovolumic Movement (IM), Systolic Peak Rapid Ejection (RE) and Diastolic Peak Rapid Filling (RF) are not commonly used in practice to calculation cardiac timing intervals, however these fiducials could be used to calculate specific timing intervals between any other fiducial points or events of the cardiac cycle. Using these CTI values via a clinician dashboard, computer application or mobile application to accurately assess valvular and ventricular mechanical delays, which indicate a variety of cardiac conditions, such as dyssynchrony, ischemic cardiovascular disease, Acute Coronary Syndrome (ACS), Myocardial Infarction (MI), Hypertrophic Cardiomyopathy (HCM), Restrictive Cardiomyopathy (RCM), Arrhythmia and common valvular diseases like Aortic Stenosis, Aortic Regurgitation, Mitral Stenosis or Mitral Regurgitation.

Using an accelerometer to measure acceleration rather than velocity directly reveals how quickly signal is changing, giving insights into the dynamics of the motion of the heart, which is more sensitive to abrupt changes in velocity such as the openings and closing of heart valves, and less sensitive to slower changing events such as blood flow. This is where analyzing SCG signals will have an advantage over PCG signals. However, combining different technology sensors can also present improved sensitivity over using single sensor technology.

The device with the sensors for measuring the signals, need not be a stand-alone device. A small device without a user interface is desired for patient comfort, where the device is equipped with a communications interface for connecting to a mobile device, tablet or computer to both send data and act as a user interface for the device. This configuration of the device and sensors lends well to patient home use cases.

Other embodiments can use sensors other than accelerometers, such as gyroscopes, phono and piezoelectric that are capable of detecting mechanical physiological movements. Which are directly applicable as an alternate technology for the embodiments.

Other embodiments can use a combination of sensor types to aid in identification and isolation of targeting cardiac events.

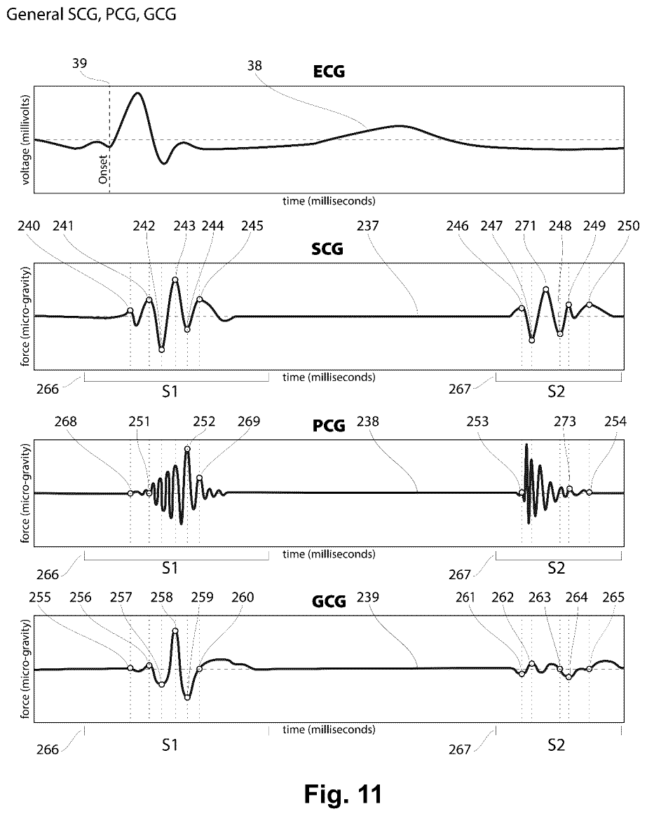

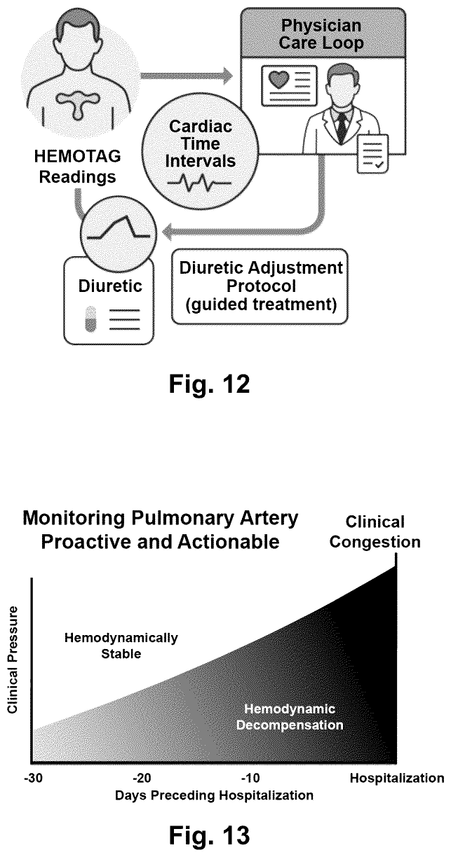

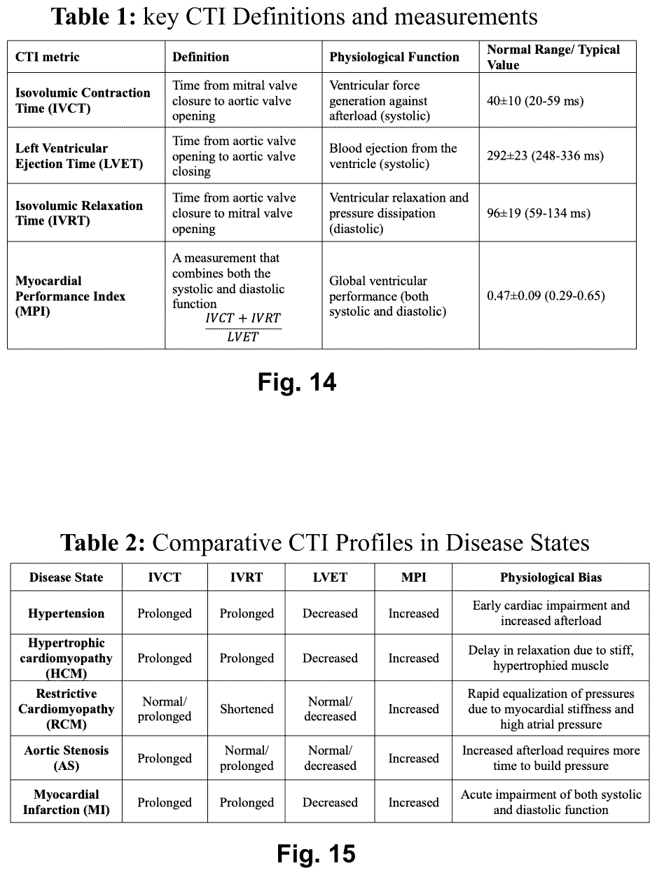

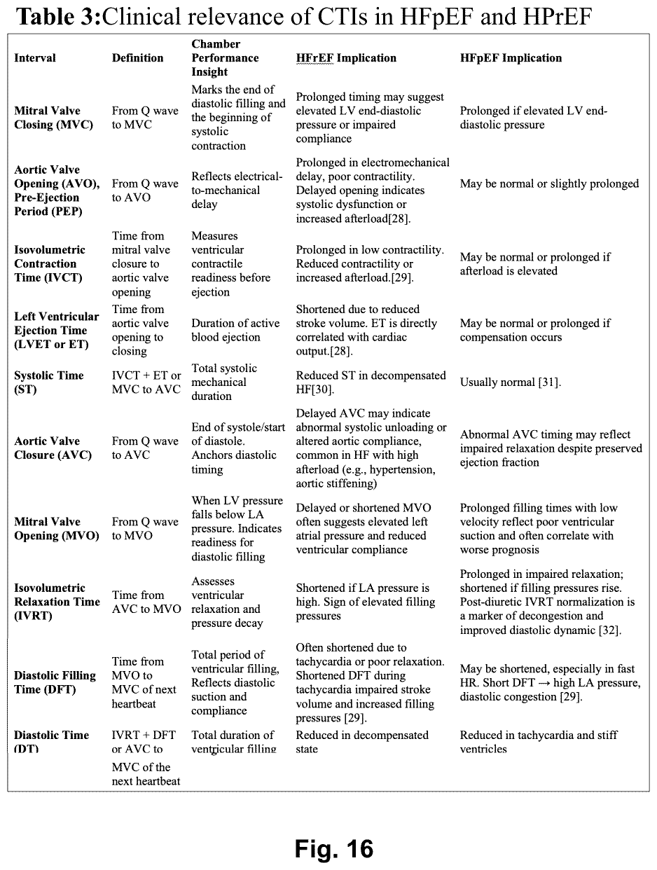

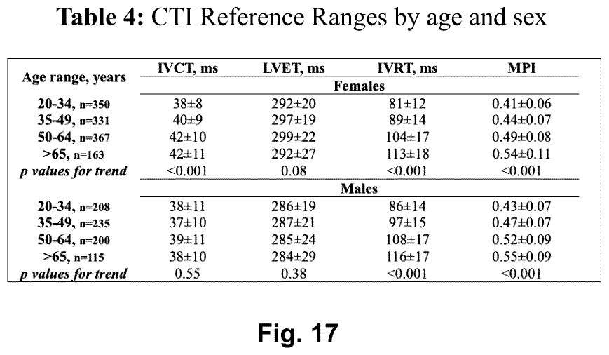

FIG. 11 illustrates three different mechanical technologies, 237 SCG (seismocardiogram) signal, 238 PCG (phonocardiogram) signal and 239 GCG (gyrocardiogram) signal showing the relationship of the events of the cardiac cycle for all three types of signals as a timed synchronized comparison. Atrial Systole (AS) is evident as 240 on FIG. 11 for SCG, 268 for PCG and 255 for GCG, with each signal having its own signature for that event of the cardiac cycle. For 240 AS on SCG on FIG. 11 is a clear peak, whereas 268 AS on PCG and 255 AS on GCG are identified by a positive and negative deflection from baseline respectively, demonstrating that although each event of the cardiac cycle can be identified across various technologies, each technology must be tuned accordingly and some technologies will be better at identification of some points than others.

FIG. 11 illustrates additional major CTIs across the different technologies, such as Mitral Close (MC) 241 251 256, Isovolumic moment (IM) 242 257, Aortic Open (AO) 243 252 258, Isotonic Contraction (IC) 244 259, Rapid Ejection (RE) 245 269 260, Aortic Close (AC) 246 253 261, Isovolumic Relaxation Point (IRP) 271, Mitral Open (MO) 249 273 264 and Rapid Filling (RF) 250 254 265. Other events of the cardiac cycle have yet to be named in any research papers, although they are clear landmarks for these events seen in FIG. 11 as 247 263 248 263.

1. A system, comprising:
a device for performing a diagnostic measurement, the device having at least two sensors for measuring time-synchronized vibrations and an electrical sensor for measuring the onset of the heartbeat, wherein the sensors are arranged in a predetermined position relative to each other;
the device further comprising a microprocessor for signal processing;
at least one computing device using phase differences to locate origin of vibrations from the at least two sensors for measuring time-synchronized vibrations.

2. The system of claim 1, where the computing device identifies fiducial points on a seismocardiogram (SCG) signal that corresponds to one or more events of the cardiac cycle.

3. The system of claim 1, where the computing device identifies fiducial points on a phonocardiogram (PCG) signal that corresponds to one or more events of the cardiac cycle.

4. The system of claim 1, where the computing device identifies fiducial points on gyrocardiogram (GCG) signal that corresponds to one or more events of the cardiac cycle.

5. The system of claim 1, where the computing device identifies fiducial points on a plurality of SCG, PCG, GCG, Photoplethysmography (PPG) and electrocardiogram (ECG) signals that correspond to at least one event of the cardiac cycle.

6. The system of claim 1, where the computing device identifies signals from valvular events of the cardiac cycle.

7. The system of claim 1, where the computing device identifies signals from arteries and veins of the cardiac cycle.

8. A system, comprising:
a device for performing a diagnostic measurement, the device having at least two sensors for measuring time-synchronized vibrations; and
at least one electrical sensor for measuring the onset of the heartbeat, wherein the at least two sensors and the at least one electrical sensor are arranged in a predetermined position relative to each other;
the device comprising a microprocessor for signal processing;
at least one computing device using phase differences to locate origin of vibrations and identifies peaks and troughs of the vibrational signals corresponding to cardiac events.

9. The system of claim 8, where the computing device matches the peaks and the troughs to the events of the cardiac cycle and calculates a time interval between at least two of the events of the cardiac cycle.

10. The system of claim 8, wherein the device resides on or within a housing that includes an adhesive pad that allows the sensors to be placed at a standardized chest position and wherein the system is configured to acquire data in a resting position and to complete the measurement within about 30 seconds.

11. The system of claim 8, wherein the system processes the synchronized waveforms to identify multi chamber cardiac time intervals, including pre ejection period (PEP), AO, AC, MO, MC, isovolumetric contraction time (IVCT), left ventricular ejection time (LVET), right ventricular ejection time (ET), STI, MPI, ventricular mechanical delay and electromechanical valvular delay.

12. An apparatus for synchronized multi modal non-invasive cardiac monitoring, comprising:
a body mountable housing; and
a plurality of sensors including a least one electrocardiogram sensor and two or more mechanical vibration sensors;
a processor coupled to the plurality of sensors; and
wherein the processor is configured to:
(a) synchronously acquire cardiac signal data from the plurality of sensors; and
(b) compute cardiac time intervals from at least one cardiac chamber.

13. The apparatus of claim 12, wherein machine-learning models utilize the multi-chamber time intervals data to estimate diseases, in patients with, cardiac dysfunction in hypertensive and ischemic cardiovascular disease, Acute Coronary Syndrome (ACS) and Myocardial Infarction (MI), Hypertrophic Cardiomyopathy, Restrictive Cardiomyopathy, Valvular Heart Disease of aortic stenosis, mitral regurgitation, Arrhythmia, and heart failure.

14. The apparatus of claim 12, wherein machine-learning models utilize the multi-chamber time intervals data to estimate diseases and wherein the estimates are trended over time to detect worsening of disease and to guide therapy.

15. The apparatus of claim 12, wherein the processor is further configured to compare the estimated intracardiac parameters against predefined thresholds and, when a threshold is exceeded, generate an alert and transmit it via the communications interface to a clinician's dashboard or a patient's mobile device.

16. The apparatus of claim 12, wherein the two or more mechanical vibration sensors are arranged to concurrently acquire waveforms from at least two different auscultation points, and the processor uses differences in timing of the first and second heart sounds and mechanical vibrations across these sites to derive chamber specific delays and events.
